(19) United States
(12) Patent Application Publication
(10) Pub. No.: US 2011/0130336 A1
(43) Pub. Date: Jun. 2, 2011

(54) METHOD OF TREATING ISCHEMIC INJURY USING APOAEQUORIN

(75) Inventor: Mark Y. Underwood, Madison, WI (US)

(73) Assignee: Quincy Bioscience, LLC, Madison, WI (US)

(21) Appl. No.: 12/905,236

(22) Filed: Oct. 15, 2010

Related U.S. Application Data
(60) Provisional application No. 61/252,344, filed on Oct. 16, 2009.

(51) Int. Cl. A61K 38/17; A61P 25/00

(52) U.S. Cl. 514/17.7

(57) ABSTRACT

Compositions containing apoaequorin and methods for their use in treating ischemic injury, particularly reduction in neuronal cell death following ischemic insult, are provided by the present invention.

### CROSS-REFERENCE TO RELATED APPLICATIONS

This application is a non-provisional utility application claiming the benefit of U.S. Application No. 61/252,344, filed Oct. 16, 2009, which is incorporated herein by reference in its entirety for all purposes.

### STATEMENT REGARDING FEDERALLY SPONSORED RESEARCH OR DEVELOPMENT

Not Applicable.

### FIELD OF THE INVENTION

This invention relates generally to methods for treatment of ischemic injury. In particular, this invention is directed to the use of apoaequorin to provide protection to neurons following an ischemic insult.

### BACKGROUND OF THE INVENTION

Neurons are continuously subjected to alterations in intracellular Ca²⁺ as a result of ongoing activity and these changes are necessary for certain normal neuronal processes to occur, however too much Ca²⁺ can be toxic (Bano et al., 2005; Choi, 1992; Lee et al., 1999). As a result, the intracellular Ca²⁺ concentration in neurons is very tightly regulated (Kristian & Siesjo, 1998). Several mechanisms enable neurons to buffer/maintain homeostasis or control cytosolic Ca²⁺ levels (Baimbridge et al., 1992; Chard et al., 1993), including calcium binding proteins (CaBPs). CaBPs confer some protection against excitotoxic insults, which would normally kill the cell if they were not present (Gary et al., 2000).

Decreased levels of CaBPs are observed with advancing age (De Jong et al., 1996; Krzywkowski et al., 1996; Moyer et al., in press; VIIIa et al., 1994), and in neurodegenerative disorders (Mattson & Magnus, 2006), including: Alzheimer's disease (H of & Morrison, 1991; Iacopino & Christakos, 1990; Mikkonen et al., 1999; Sutherland et al., 1993); Parkinson's disease (Iacopino & Christakos, 1990); and ischemia (Yenari et al., 2001).

During ischemia, neurons are subjected to excess Ca²⁺ influx triggering a cascade of events leading to cell death (Choi, 1992). Treatments aimed at minimizing Ca²⁺ toxicity during ischemia have been administered before an ischemic insult, with positive results. For example, Yenari et al. (2001) treated animals with calbindin prior to ischemia and found that the pre-treatment with calbindin resulted in fewer dead neurons. Fan et al. (2007) found a smaller infarct volume, better behavioral recovery, and decreased apoptosis in rats pre-treated with calbindin.

However, there is a long recognized, unmet need in the field to identify compositions and treatments that provide neuroprotection when provided after an ischemic insult. It can be appreciated that the pre-treatment of a subject is generally not feasible, as subjects generally seek medical aid only after an ischemic event has occurred. Accordingly, compositions and methods applied subsequent to an ischemic event and which improve neuron survival are highly sought after in this field.

### SUMMARY OF THE INVENTION

In a first aspect, the present invention provides a composition for reducing neuronal cell death following an ischemic insult in a subject, comprising: (a) an effective amount of apoaequorin; and (b) an acceptable carrier. The composition is preferably in the form of an injectable dosage.

In a second aspect, the invention is directed to a method for reducing neuronal cell death following an ischemic insult in a subject. Such a method includes the step of administering to a subject that has suffered an ischemic insult an effective amount of apoaequorin. Administration is preferably to the central nervous system (CNS) of the subject, more preferably the brain of the subject. The preferred route of administration is by injection of an injectable dosage of apoaequorin.

The invention further encompasses the use of apoaequorin for the manufacture of a medicament for reducing neuronal cell death following an ischemic insult in a subject administered the medicament as well as apoaequorin for use in the treatment of neuronal cell injury following an ischemic insult in a subject.

Other objects, features and advantages of the present invention will become apparent after review of the specification and claims.

### BRIEF DESCRIPTION OF THE DRAWINGS

FIG. 1 illustrates that apoaequorin injected prior to ischemia is neuroprotective. “AQ” refers to apoaequorin throughout the figures.

FIG. 2 shows the neuroprotective effects of apoaequorin last less than 48 hours.

FIG. 3 illustrates that apoaequorin is detected in hippocampus at 24 hours, but not 48 hrs.

FIG. 4 shows that apoaequorin administered immediately after ischemia is neuroprotective.

### DETAILED DESCRIPTION OF THE INVENTION

Before the present materials and methods are described, it is understood that this invention is not limited to the particular methodology, and materials described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Aequorin is a photo-protein originally isolated from luminescent jellyfish and other marine organisms. The aequorin complex comprises a 22,285-dalton apoaequorin protein, molecular oxygen and the luminophore coelenterazine. When three Ca²⁺ ions bind to this complex, coelenterazine is oxidized to coelentermide, with a concomitant release of carbon dioxide and blue light. Aequorin is not exported or secreted by cells, nor is it compartmentalized or sequestered within cells. Accordingly, aequorin measurements have been used to detect Ca²⁺ changes that occur over relatively long periods. In several experimental systems, aequorin's luminescence was detectable many hours to days after cell loading. It is further known that aequorin also does not disrupt cell functions or embryo development.

Because of its Ca²⁺-dependent luminescence, the aequorin complex has been extensively used as an intracellular Ca²⁺ indicator. Aequorea victoria aequorin has been specifically used to: (1) analyze the secretion response of single adrenal chromaffin cells to nicotinic cholinergic agonists; (2) clarify the role of Ca²⁺ release in heart muscle damage; (3) demonstrate the massive release of Ca²⁺ during fertilization; (4) study the regulation of the sarcoplasmic reticulum Ca²⁺ pump expression in developing chick myoblasts; and (5) calibrate micropipets with injection volumes of as little as three picoliters.

Apoaequorin has an approximate molecular weight of 22 kDa. Apoaequorin can be used to regenerate aequorin by reducing the disulfide bond in apoaequorin. The calcium-loaded apoaequorin retains the same compact scaffold and overall folding pattern as unreacted photoproteins containing a bound substrate.

Conventional purification of aequorin from the jellyfish Aequorea victoria requires laborious extraction procedures and sometimes yields preparations that are substantially heterogeneous or that are toxic to the organisms under study. Two tons of jellyfish typically yield approximately 125 mg of the purified photoprotein. In contrast, recombinant aequorin is preferably produced by purifying apoaequorin from genetically engineered Escherichia coli, followed by reconstitution of the aequorin complex in vitro with pure coelenterazine. Apoaequorin useful in the present invention has been described and is commercially-obtainable through purification schemes and/or syntheses known to those of skill in the art. S. Inouye, S. Zenno, Y. Sakaki, and F. Tsuji. High level expression and purification of apoaequorin. (1991) Protein Expression and Purification 2, 122-126.

As used herein, the term “treating” includes preventative as well as disorder remittent treatment. As used herein, the terms “reducing”, “alleviating”, “suppressing” and “inhibiting” have their commonly understood meaning of lessening or decreasing. As used herein, the term “progression” means increasing in scope or severity, advancing, growing or becoming worse. As used herein, the term “recurrence” means the return of a disease after a remission.

As used herein, the term “administering” refers to bringing a patient, tissue, organ or cell in contact with apoaequorin. As used herein, administration can be accomplished in vitro, i.e., in a culture or other tissue preparation outside the living organism, or in vivo, i.e., in cells or tissues of living organisms, for example, humans. In preferred embodiments, the present invention encompasses administering the compositions useful in the present invention to a patient or subject. A “patient” or “subject”, used equivalently herein, refers to a mammal, preferably a human, that either: (1) has a disorder remediable or treatable by administration of apoaequorin; or (2) is susceptible to a disorder that is preventable by administering apoaequorin.

As used herein, the terms “effective amount” and “therapeutically effective amount” refer to the quantity of active agents sufficient to yield a desired therapeutic response without undue adverse side effects such as toxicity, irritation, or allergic response. The specific “effective amount” will, obviously, vary with such factors as the particular condition being treated, the physical condition of the patient, the type of animal being treated, the duration of the treatment, the nature of concurrent therapy (if any), and the specific formulations employed and the structure of the compounds or its derivatives. In this case, an amount would be deemed therapeutically effective if it resulted in the reduction of neuronal cell death following an ischemic insult/injury. The optimum effective amounts can be readily determined by one of ordinary skill in the art using routine experimentation.

During ischemia, the deprivation of blood flow and oxygen to the brain results in excessive calcium influx through glutamate receptors, which can rapidly trigger cell death. One way neurons protect themselves from the toxic effects of calcium is to buffer the calcium with calcium binding proteins (CaBPs). Previous work has demonstrated that hippocampal neurons expressing the CaBP calbindin-D28k are better able to withstand an excitotoxic insult than neurons lacking calbindin. The inventors have been investigating the feasibility of regulating calcium levels during ischemia by replenishing CaBPs. Apoaequorin is a 22 kDa CaBP isolated from the coelenterate Aequorea victoria. AQ has been used for years as an auto-fluorescent indicator for monitoring calcium levels and has been shown to be safe and well tolerated by cells. The present studies were designed to test the hypothesis that intrahippocampal infusion of apoaequorin can protect neurons from an ischemic insult. Rats were stereotaxically implanted with bilateral cannula (in the CA1 region of the dorsal hippocampus) under aseptic conditions. After recovery, rats received an intrahippocampal infusion of AQ (0.4%, 1%, or 4%) in one hemisphere and artificial CSF (aCSF) in the other (0.5 μl/min for 1 min). Twenty-four or 72 hours following the infusion, coronal brain slices (400 μm) were cut with a vibratome. Slices were maintained in oxygenated aCSF for 1 hr. They were then subjected to a 5-min oxygen-glucose deprivation (OGD), returned to oxygenated aCSF (with 0.2% Trypan blue) for a 30-min reperfusion and then rinsed in oxygenated aCSF. All slice experiments were carried out at 35° C. Slices were then fixed, cryoprotected, sub-sectioned (40 μm), mounted, and coverslipped. An individual blind to treatment group counted the number of trypan blue stained (dead) CA1 neurons, and the number of dead cells in the AQ-treated hemisphere was compared to the aCSF-treated hemisphere to calculate a percent rescue. Apoaequorin treatment prior to OGD resulted in significantly fewer Trypan blue stained CA1 neurons relative to control. In addition, the rats injected with 4% apoaequorin had more rescue (58±12%) than those injected with 0.4% apoaequorin (37±20%). However, when OGD was initiated 72 hours after 4% apoaequorin infusion, no neuroprotection was noted. These data support the hypothesis that apoaequorin may be an effective neurotherapeutic against ischemia when administered within 24 hours prior to an ischemic insult. In addition, and of great importance, the present inventors have obtained further data that support the hypothesis that delivery of apoaequorin is neuroprotective when administered following an ischemic insult.

Accordingly, the present invention provides a composition for reducing neuronal cell death following an ischemic insult in a subject, comprising: (a) an effective amount of apoaequorin; and (b) an acceptable carrier. The composition is preferably in the form of an injectable dosage.

In a second aspect, the invention is directed to a method for reducing neuronal cell death following an ischemic insult in a subject. Such a method includes the step of administering to a subject that has suffered an ischemic insult an effective amount of apoaequorin. Administration is preferably to the central nervous system (CNS) of the subject, more preferably the brain of the subject. The preferred route of administration is by injection to the subject's CNS/brain.

The invention further encompasses the use of apoaequorin for the manufacture of a medicament for reducing neuronal cell death following an ischemic insult in a subject administered the medicament as well as apoaequorin for use in the treatment of neuronal cell injury following an ischemic insult in a subject.

Compositions according to the present invention include liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, or hydrogels, or onto liposomes, microemulsions, micelles, lamellar or multilamellar vesicles, erythrocyte ghosts or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

Also encompassed by the invention are methods of administering particulate compositions coated with polymers (e.g., poloxamers or poloxamines). Other embodiments of the compositions incorporate particulate forms protective coatings, protease inhibitors or permeation enhancers for various routes of administration, including parenteral, pulmonary, nasal and oral. In certain embodiments, the composition is administered parenterally, paracancerally, transmucosally, intramuscularly, intravenously, intradermally, subcutaneously, intraperitonealy, intraventricularly, intracranially or intratumorally.

Further, as used herein, “pharmaceutically acceptable carriers” are well known to those skilled in the art and include, but are not limited to, 0.01-0.1M and preferably 0.05M phosphate buffer or 0.9% saline. Additionally, such pharmaceutically acceptable carriers may be aqueous or non-aqueous solutions, suspensions and emulsions. Examples of non-aqueous solvents are propylene glycol, polyethylene glycol, vegetable oils such as olive oil and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions administrable according to the invention include formulation in lipophilic depots (e.g., fatty acids, waxes, oils). Also comprehended by the invention are particulate compositions coated with polymers (e.g., poloxamers or poloxamines) and the compound coupled to antibodies directed against tissue-specific receptors, ligands or antigens or coupled to ligands of tissue-specific receptors.

Other embodiments of the compositions administered according to the invention incorporate particulate forms, protective coatings, protease inhibitors or permeation enhancers for various routes of administration, including parenteral, pulmonary, nasal, ophthalmic and oral.

Chemical entities modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds. Such modifications may also increase the chemical entities solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-entity abducts less frequently or in lower doses than with the unmodified entity.

In yet another method according to the invention, the composition can be delivered in a controlled release system. For example, the agent may be administered using intravenous infusion, an implantable osmotic pump, a transdermal patch, liposomes, or other modes of administration. In one embodiment, a pump may be used. In another embodiment, polymeric materials can be used. In yet another embodiment, a controlled release system can be placed in proximity to the therapeutic target, i.e., the brain, thus requiring only a fraction of the systemic dose.

As disclosed herein, apoaequorin is particularly useful when formulated in the form of a pharmaceutical injectable dosage, including apoaequorin in combination with an injectable carrier system. As used herein, injectable and infusion dosage forms (i.e., parenteral dosage forms) include, but are not limited to, liposomal injectables or a lipid bilayer vesicle having phospholipids that encapsulate an active drug substance. Injection includes a sterile preparation intended for parenteral use.

Five distinct classes of injections exist as defined by the USP: emulsions, lipids, powders, solutions and suspensions. Emulsion injection includes an emulsion comprising a sterile, pyrogen-free preparation intended to be administered parenterally. Lipid complex and powder for solution injection are sterile preparations intended for reconstitution to form a solution for parenteral use. Powder for suspension injection is a sterile preparation intended for reconstitution to form a suspension for parenteral use. Powder lyophilized for liposomal suspension injection is a sterile freeze dried preparation intended for reconstitution for parenteral use that is formulated in a manner allowing incorporation of liposomes, such as a lipid bilayer vesicle having phospholipids used to encapsulate an active drug substance within a lipid bilayer or in an aqueous space, whereby the formulation may be formed upon reconstitution. Powder lyophilized for solution injection is a dosage form intended for the solution prepared by lyophilization (“freeze drying”), whereby the process involves removing water from products in a frozen state at extremely low pressures, and whereby subsequent addition of liquid creates a solution that conforms in all respects to the requirements for injections. Powder lyophilized for suspension injection is a liquid preparation intended for parenteral use that contains solids suspended in a suitable fluid medium, and it conforms in all respects to the requirements for Sterile Suspensions, whereby the medicinal agents intended for the suspension are prepared by lyophilization. Solution injection involves a liquid preparation containing one or more drug substances dissolved in a suitable solvent or mixture of mutually miscible solvents that is suitable for injection. Solution concentrate injection involves a sterile preparation for parenteral use that, upon addition of suitable solvents, yields a solution conforming in all respects to the requirements for injections. Suspension injection involves a liquid preparation (suitable for injection) containing solid particles dispersed throughout a liquid phase, whereby the particles are insoluble, and whereby an oil phase is dispersed throughout an aqueous phase or vice-versa. Suspension liposomal injection is a liquid preparation (suitable for injection) having an oil phase dispersed throughout an aqueous phase in such a manner that liposomes (a lipid bilayer vesicle usually containing phospholipids used to encapsulate an active drug substance either within a lipid bilayer or in an aqueous space) are formed. Suspension sonicated injection is a liquid preparation (suitable for injection) containing solid particles dispersed throughout a liquid phase, whereby the particles are insoluble. In addition, the product may be sonicated as a gas is bubbled through the suspension resulting in the formation of microspheres by the solid particles.

The parenteral carrier system includes one or more pharmaceutically suitable excipients, such as solvents and co-solvents, solubilizing agents, wetting agents, suspending agents, thickening agents, emulsifying agents, chelating agents, buffers, pH adjusters, antioxidants, reducing agents, antimicrobial preservatives, bulking agents, protectants, tonicity adjusters, and special additives. The tolerable dosage for administration to animals, including humans, is from about 0.001 mg/kg to about 1000 mg/kg.

The invention will be more fully understood upon consideration of the following non-limiting Examples.

### EXAMPLES

### Materials and Methods

Animals. Fifty-three male F344 adult rats (mean age=4.6±0.1 mo.) were used. Rats were kept on a 14/10-hour day/night cycle with free access to food and water.

Surgery. Rats were anesthetized and mounted on a stereotaxic apparatus. Under aseptic conditions, the scalp was incised and retracted to the side, and the head was leveled between bregma and lambda. Each rat was prepared with bilateral stainless steel guide cannulae aimed at the dorsal hippocampus (dhpc) using sterotaxic coordinates (3.5 mm posterior, ±2.6 mm lateral, 3.0 mm ventral) relative to bregma. Cannulae were secured to the skull with stainless steel screws and epoxy. A stainless steel cap remained in place when the rats were not being injected to prevent the guide cannulae from becoming occluded.

Drugs and Infusions. Rats were given an infusion of 0, 0.4, 1, or 4% apoaequorin in one hemisphere and aCSF in the other hemisphere 1, 24, 48, or 72 hours prior to decapitation. To facilitate neuronal uptake of apoaequorin, 6% DMSO was added. All rats received bilateral infusions (0.5 μl/side) over 60 seconds and the injection cannulae remained in place for an additional 2 min to ensure diffusion. The infusion cannulae were cut to extend 0.5 mm beyond the guide cannulae.

Slice Preparation. 400 μm thick slices were prepared using standard procedures (Moyer & Brown, 1998). Following slice recovery, in vitro ischemia was induced by transferring slices to fructose-CSF (glucose replaced with fructose and bubbled with 95% N2-5% CO2 instead of a 95% O2-5% CO2). The slices were in the ischemia condition for 5 minutes, and were then returned to oxygenated aCSF that contained 0.2% Trypan blue for 30 minutes. Trypan blue readily penetrates dead cells and stains them blue while leaving living cells unstained (DeRenzis & Schechtman, 1973). The slices were rinsed in oxygenated room temperature aCSF twice then fixed in 10% neutral buffered formalin overnight in the refrigerator. Slices were then cryoprotected, cut on a cryostat (40 μm), and mounted onto subbed slides.

Post-ischemia Apoaequorin. Slices were prepared as listed above and were transferred to interface chambers for one hour of recovery. In vitro ischemia was induced by switching the perfusion solution to fructose-CSF bubbled with 95% N2-5% CO2 and the chambers were filled with 95% N2-5% CO2 gas for 5 minutes. Immediately following in vitro ischemia, perfusion solution and chambers were returned to oxygenated aCSF and 4% apoaequorin or aCSF (25 μL) was pipetted onto each slice. Five minutes later, the slices were removed from the interface chambers and stained with 0.2% Trypan blue as above.

Cell Counts. Slices were examined under an Olympus microscope (equipped with a digital camera) at 10×, and pictures were taken. Trypan blue stained neurons within CA1 (about an 800 μm section) were counted by an experimenter blind to experimental conditions. Statistical analyses were performed using Statview (v 5.0; SAS Institute, Inc., Cary, N.C.). An ANOVA was used to evaluate a drug effect. Asterisk indicates p<0.05 from 0% apoaequorin.

Western Blots. Rats were given a bilateral injection of apoaequorin and sacrificed at one of the following time points: 1 hr, 24 hr, 48 hr, or 72 hr. Brains were removed, rapidly frozen, and stored at −80° C. The dhpc and ventral hippocampus (vhpc) were dissected out and homogenized separately. Samples were centrifuged and the supernatant removed and measured using a Bradford protein assay kit (Bio-Rad). Protein samples were normalized and loaded for SDS-PAGE (9%). Proteins were transferred onto membranes using a semidry transfer apparatus (Bio-Rad). Membranes were then incubated in blocking buffer (2 hr), primary antibody (overnight at 4° C.; 1:200 mouse anti-aequorin [Chemicon], and secondary antibody (90 min; 1:5000 anti-mouse [Santa Cruz Biotechnology]). Membranes were then washed, placed in a chemiluminescence solution (Santa Cruz Biotechnology), and exposed to autoradiagraphic film (Hyperfilm MP). Images were taken and densitometry was performed using NIH Image J Software by an experimenter blind to lane conditions. A percentage of control score was derived for each rat by dividing each animal's relative optical density score by the mean of the 1 hr time point.

### Example 1

### The Calcium Binding Protein Apoaequorin is Neuroprotective when Injected into Hippocampus Prior to Ischemia

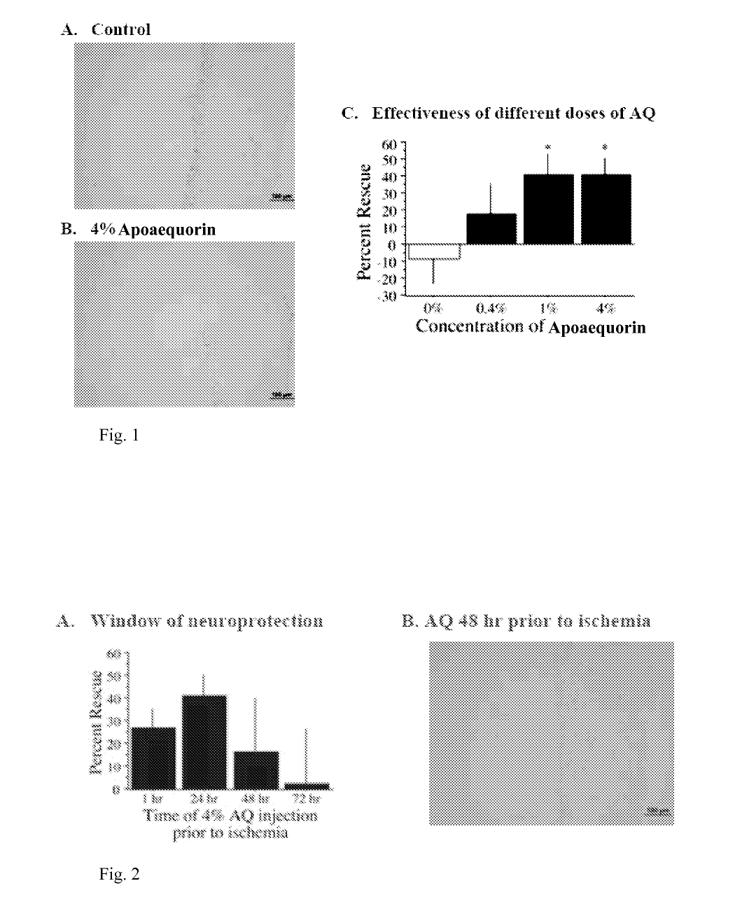

FIG. 1 illustrates that there were fewer dead cells in area CA1 of the hippocampus in the aequorin-injected hemisphere compared to the control-injected hemisphere

### Example 2

### The Neuroprotective Effect of Apoaequorin is Time-Dependent

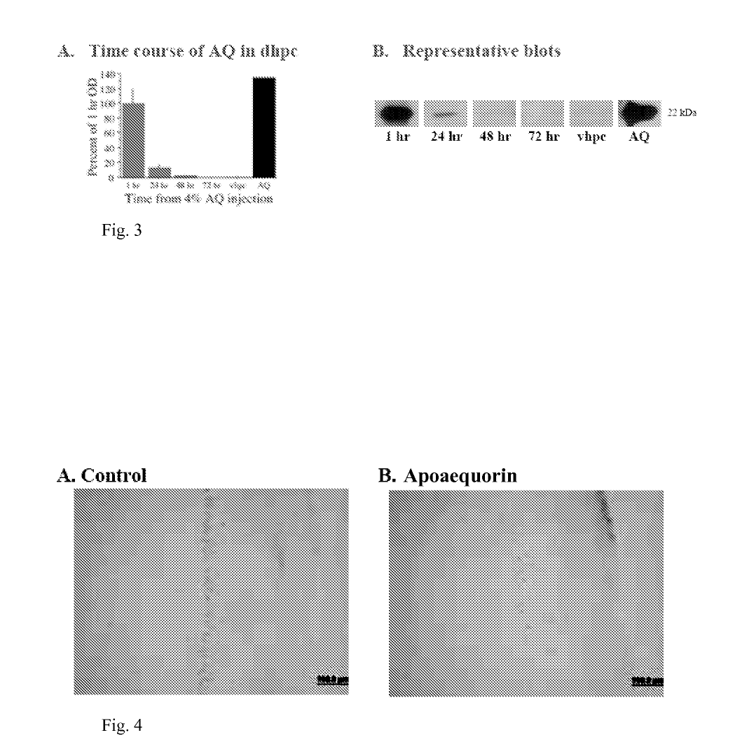

FIGS. 2 and 3 show that when injected 48 or 72 hrs prior to ischemia, apoaequorin was no longer neuroprotective.

### Example 3

### Apoaequorin is Neuroprotective when Administered Post-Ischemia

FIG. 4 illustrates data showing there were fewer dead cells in slices given apoaequorin immediately after 5 min OGD compared to slices given aCSF.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

Baimbridge, K. G., Celio, M. R., & Rogers, J. H. (1992). Calcium-binding proteins in the nervous system. Trends in Neuroscience, 15 (8), 303-308
Bano, D., Young, K. W., Guerin, C. J., Lefeuvre, R., Rothwell, N. J., Naldini, L., et al. (2005). Cleavage of the plasma membrane Na+/Ca2+ exchanger in excitotoxicity. Cell, 120 (2), 275-285.
Chard, P. S., Bleakman, D., Christakos, S., Fullmer, C. S., & Miller, R. J. (1993). Calcium buffering properties of calbindin D28k and parvalbumin in rat sensory neurones. Journal of Physiology, 472, 341-357.
Choi, D. W. (1992). Excitotoxic cell death. Journal of Neurobiology, 23 (9), 1261-1276.
DeRenzis, F. A., & Schechtman, A. (1973). Staining by neutral red and Trypan blue in sequence for assaying vital and nonvital cultured cells. Stain Technology, 48 (3), 135-136.
De Jong, G. I., Naber, P. A., Van der Zee, E. A., Thompson, L. T., Disterhoft, J. F., & Luiten, P. G. M. (1996). Age-related loss of calcium binding proteins in rabbit hippocampus. Neurobiology of Aging, 17 (3), 459-465.
Fan, Y., Shi, L., Gu, Y., Zhao, Y., Xie, J., Qiao, J., et al. (2007). Pretreatment with PTD-calbindin D 28k alleviates rat brain injury induced by ischemia and reperfusion. Journal of Cerebral Blood Flow and Metabolism, 27 (4), 719-728.
Gary, D. S., Sooy, K., Chan, S. L., Christakos, S., & Mattson, M. P. (2000). Concentration- and cell type-specific effects of calbindin D28k on vulnerability of hippocampal neurons to seizure-induced injury. Brain Research. Molecular Brain Research, 75 (1), 89-95.
H of, P. R., & Morrison, J. H. (1991). Neocortical neuronal subpopulations labeled by a monoclonal antibody to calbindin exhibit differential vulnerability in Alzheimer's disease. Experimental Neurology, 111, 293-301.
Iacopino, A. M., & Christakos, S. (1990). Specific reduction of calcium-binding protein (28-kilodalton calbindin-D) gene expression in aging and neurodegenerative diseases. Proceedings of the National Academy of Sciences (USA), 87, 4078-4082.
Kristian, T., & Siesjo, B. K. (1998). Calcium in ischemic cell death. Stroke, 29 (3), 705-718.
Krzywkowski, P., Potier, B., Billard, J. M., Dutar, P., & Lamour, Y. (1996). Synaptic mechanisms and calcium binding proteins in the aged rat brain. Life Sciences, 59 (5/6), 421-428.
Lee, J. M., Zipfel, G. J., & Choi, D. W. (1999). The changing landscape of ischaemic brain injury mechanisms. Nature, 399 (6738 Suppl), A7-14.
Mattson, M. P., & Magnus, T. (2006). Ageing and neuronal vulnerability. Nature Reviews. Neuroscience, 7 (4), 278-294.
Mikkonen, M., Alafuzoff, I., Tapiola, T., Soininen, H., & Niettinen, R. (1999). Subfield- and layer-specific changes in parvalbumin, calretinin and calbindin-D28K immunoreactivity in the entorhinal cortex in Alzheimer's disease. Neuroscience, 92 (2), 515-532.
Moyer, J. R., Jr., & Brown, T. H. (1998). Methods for whole-cell recording from visually preselected neurons of perirhinal cortex in brain slices from young and aging rats. Journal of Neuroscience Methods, 86 (1), 35-54.
Moyer, J. R., Jr., Furtak, S. C., McGann, J. P., & Brown, T. H. (in press). Aging-related changes in calcium binding proteins in rat perirhinal cortex. Neurobiology of Aging.
Sutherland, M. K., Wong, L., Somerville, M. J., Yoong, L. K. K., Bergeron, C., Parmentier, M., et al. (1993). Reduction of calbindin-28k mRNA levels in Alzheimer as compared to Huntington hippocampus. Brain Research. Molecular Brain Research, 18 (1-2), 32-42.
Villa, A., Podini, P., Panzeri, M. C., Racchetti, G., & Meldolesi, J. (1994). Cytosolic Ca2+ binding proteins during rat brain ageing: loss of calbindin and calretinin in the hippocampus, with no change in the cerebellum. European Journal of Neuroscience, 6, 1491-1499.
Yenari, M. A., Minami, M., Sun, G. H., Meier, T. J., Kunis, D. M., McLaughlin, J. R., et al. (2001). Calbindin d28k overexpression protects striatal neurons from transient focal cerebral ischemia. Stroke, 32 (4), 1028-1035.

What is claimed is:

1. A composition for reducing neuronal cell death following an ischemic insult in a subject, comprising: (a) an effective amount of apoaequorin; and (b) an acceptable carrier.

2. The composition according to claim 1, wherein said composition is in the form of an injectable dosage.

3. A method for reducing neuronal cell death following an ischemic insult in a subject, comprising administering to a subject that has suffered an ischemic insult an effective amount of apoaequorin.

4. The method according to claim 3, wherein administration is to the central nervous system of said subject.

5. The method according to claim 3, wherein administration is to the brain of said subject.

6. The method according to claim 3, wherein administration is by injection of an injectable dosage of said apoaequorin.
